(19) United States
(12) Patent Application Publication
(10) Pub. No.: US 2022/0087916 A1
(43) Pub. Date: Mar. 24, 2022

(54) ORAL RINSE COMPOSITION, ORAL CLEANSING KIT, METHOD OF CLEANSING ORAL CAVITY AND METHOD OF MAKING ORAL RINSE COMPOSITION

(71) Applicant: Medline Industries, LP, Northfield, IL (US)

(72) Inventors: Joseph Bryzek, JR., Kenosha, WI (US); Sharbel Maalouf, Pleasant Prairie, WI (US); Clare Walsh, Riverside, IL (US)

(21) Appl. No.: 17/474,785

(22) Filed: Sep. 14, 2021

Related U.S. Application Data
(60) Provisional application No. 63/082,859, filed on Sep. 24, 2020.

(51) Int. Cl. A61K 8/49; A61Q 11/00; A61K 8/34

(52) U.S. Cl. CPC A61K 8/4926; A61K 2800/74; A61K 8/345; A61Q 11/00

(57) ABSTRACT

An oral rinse composition includes a solution containing an active ingredient, a humectant, flavoring, and water. An oral cleansing kit includes a container and an oral rinse composition in the container. A method of cleansing an oral cavity includes applying an oral rinse composition to an oral cavity. A method of making an oral rinse composition includes combining a humectant, flavoring, water, and an active ingredient.

### TECHNICAL FIELD

The disclosure generally relates to an oral rinse composition having anti-microbial and moisturizing properties, a kit including an oral rinse composition, and methods of using and making an oral rinse composition.

### BACKGROUND

In hospital and other health care settings, maintaining a patient's oral hygiene is thought to reduce incidence of infections such as pneumonia in addition to avoiding oral maladies such as caries or gingivitis. With intubated or unconscious patients, oral hygiene is provided by hospital staff. Conscious patients in a hospital or institutional setting may lack concern or motivation to maintain their own oral hygiene, thus also requiring staff intervention.

Oral rinse products for use with hospitalized or other institutionalized patients are known. A conventional medicinal oral rinse product includes about 1.5% hydrogen peroxide (H₂O₂). Products including hydrogen peroxide can have a bad taste, discouraging use by conscious patients. Oral hygiene products including hydrogen peroxide can also have foaming properties, which may alarm family members who observe foaming during oral cleansing of a hospitalized relative.

Also, products including hydrogen peroxide tend to have undesirable drying properties when applied in the oral cavity. It is sometimes necessary to first apply an oral cleanser including hydrogen peroxide with no moisturizer and then applying a separate moisturizer. Moisturizer is sometimes supplied in a packet as a gel and applied to the oral cavity using a swab. This requires a second step in the oral care routine, adding time to the cleansing process.

### DETAILED DESCRIPTION

Hydrogen peroxide has conventionally been used as a preservative. It has been found that hydrogen peroxide contributes to degradation of other ingredients of an oral rinse composition, rather than functioning as a preservative.

It has also been found that an oral rinse composition that contains an active agent and glycerin as a humectant can simultaneously provide antimicrobial/ anti-gingivitis properties, good taste to the consumer, desirable stability, and a moisturizing function when cleaning, especially when used in the absence or substantial absence of hydrogen peroxide. The oral rinse composition can be formulated with more moisturizer than is present in a conventional packaged moisturizing gel. Such an oral rinse composition can advantageously facilitate simultaneous cleansing and moisturizing of an oral cavity in a single step. While additional moisturizer could be added in a subsequent step, this often is not necessary.

An oral rinse composition prepared in accordance with some embodiments of the present disclosure can include cetylpyridinium chloride or another active ingredient, glycerin in an amount ranging from 15.00 to 45.00% w/v based on a total volume of the oral rinse composition; flavoring; and water in a minimum amount of 40% w/v based on a total volume of the oral rinse composition. The oral rinse composition should comprise essentially no polyhydric alcohol other than glycerin and essentially no amphoteric surfactant. The cetylpyridinium chloride should be used in any suitable amount, such as an amount ranging from 0.045 to 0.105% w/v based on a total volume of the oral rinse composition. The oral rinse compositions described herein further include an additional moisturizer, a flavoring, a buffering agent, a pH adjuster, a surfactant, a wetting agent, a solubilizing agent, a sweetener, and any other functional ingredient. Any one or more ingredients of the oral rinse composition can meet a U.S. pharmacopeia monograph designated as “USP-NF,” “NF,” or “USP.”

The present disclosure also provides oral rinse methods and methods for preparing an oral rinse composition, as well as kits that include the oral rinse composition.

The active ingredient can have one or more useful functions such as anti-septic, anti-microbial, anti-viral, anti-bacterial, anti-plaque, anti-gingivitis, analgesic, anti-inflammatory, anti-fungal, and anti-cavity functions. Examples of useful active ingredients include any one or more of benzydamine, betamethasone, cetylpyridinium chloride, chlorhexidine gluconate, eucalyptol, eugenol, enzymes such as lactoferrin, lactoperoxidase, lysozyme, etc., fluoride salts such as sodium fluoride, hexetidine, hydrogen peroxide, iodine, lidocaine, menthol, methyl salicylate, nystatin, phenol, potassium oxalate, povidone, tetracycline, thymol, triclosan, and xylocaine. For example, the oral rinse composition can comprise a solution including cetylpyridinium chloride in an amount sufficient for the compound to function as an anti-microbial agent, an anti-plaque agent, and/or an anti-gingivitis agent. In some embodiments, the oral rinse composition can include cetylpyridinium chloride in amounts ranging from 0.025 to 0.1% w/v, 0.04 to 0.17% w/v, 0.045 to 0.16% w/v, 0.045 to 0.105% w/v, 0.045 to 0.1% w/v, 0.05 to 0.15% w/v, 0.06 to 0.14% w/v, 0.07 to 0.13% w/v, 0.08 to 0.12% w/v, 0.09 to 0.11% w/v, 0.095 to 0.105% w/v, or about 0.10% w/v, all percentages expressed as w/v (weight cetylpyridinium chloride per 100 ml of oral rinse composition). The oral rinse cetylpyridinium chloride ingredient may meet U.S. pharmacopeia monograph USP-NF for the compound, the entire contents of which are incorporated herein by reference.

In other embodiments, not mutually exclusive with respect to the above, cetylpyridinium chloride may be present in an amount effective to reduce activity of any one of more of

staphylococcus aureas by at least 99M %,

porphyromonas gingivalis by at least 99,9998%,

klebsiella pneumoniae by at least 99.9%,

streptococcus mutans by at least 99.9998%,

prevatella intermedia by at least 9999992%,

tannerella forsythia by at least 99.9994%,

candida albicans by at least 99,6 ?i,

fusobacterium nucleatum by at least 99,9991 t.),

actinomyces viscosus by at least 99.9998%,

all reductions in activity being upon contact with the oral rinse composition for 30 seconds in vitro. Efficacy may be evaluated according to ASTM E2315-16, Standard Guide for Assessment of Antimicrobial Activity Using a Time-Kill Procedure, the entire contents of which are incorporated herein by reference.

The oral rinse composition can contain essentially no or no hydrogen peroxide. In some embodiments, an oral rinse composition contains less than 0.01 w/v of hydrogen peroxide, based on a total volume of the oral rinse composition. By minimizing or eliminating hydrogen peroxide from an oral rinse composition, it is possible to reduce problems of foaming, bad taste, and instability caused by hydrogen peroxide in conventional products.

The oral rinse composition can generally comprise a solution including any useful amount of one more humectants in an amount sufficient to moisturize an oral cavity rinsed with the oral rinse composition. Examples of useful humectants include any one or more of butylene glycol, glycerin, polyethylene glycol, propylene glycol, sorbitol, trimethyl glycine, xylitol, etc. The humectant can be present in an amount ranging from 5.00 to 90.00% w/v, 10.00 to 80.00% w/v, 10.00 to 50.00% w/v, 12.00 to 30.00% w/v, 15.00 to 25.00% w/v, 15.00 to 45.00% w/v, 18.00 to 22.00% w/v, 19.00 to 21.00% w/v, or about 20.00% w/v, all percentages again expressed as w/v (weight cetylpyridinium chloride per 100 ml of oral rinse composition). Generally, the oral rinse composition should include glycerin as a humectant.

Although in some cases the composition can include other polyhydric or monohydric alcohols, the oral rinse composition can comprise essentially no or no alcohol other than glycerin. Some embodiments of an oral rinse composition can comprise essentially no or no polyhydric alcohol other than glycerin. Some aspects of an oral rinse composition can comprise essentially no or no monohydric alcohol, or even essentially no or no ethanol.

The oral rinse composition further comprises a solvent. The solvent may be purified water, which may be present in a minimum amount of 40% w/v. The water may be present in amounts ranging from 50.00 to 80.00% w/v, 55.00 to 85.00% w/v, 60.00 to 98.00% w/v, 70.00 to 97.00% w/v, 75.00 to 95.00% w/v, 80.00 to 90.00% w/v, 84.00 to 85.00% w/v, about 84.20% w/v. It is further contemplated that the an oral rinse composition in other embodiments can include essentially no or no water and more than 80.00% w/v, more than 84.00% w/v, more than 85.00% w/v, more than 90.00% w/v, or more than 90.00% w/v of glycerin. Preferably, the oral rinse composition is in the form of a solution and not a suspension or dispersion, although it is contemplated in some embodiments that the composition may take the form of a suspension or dispersion.

The oral rinse composition can include a buffering agent, a pH adjuster, or both. Examples of useful buffering agents and pH adjusters include any one or more of organic acids such as acetic acid, benzoic acid, citric acid, etc., and salts thereof such as sodium citrate, sodium benzoate, etc.; phosphoric acid and salts thereof such as dicalcium phosphate, disodium phosphate, etc. In some embodiments, oral rinse composition can include sodium citrate as a buffering agent and citric acid as a buffering agent/pH adjuster.

The oral rinse composition can have any pH that is useful, safe for contact, and compatible with mucosal membranes in an oral cavity. An oral rinse composition can have a slightly acidic pH ranging from 2.5 to 7, 3 to 6, 3.5 to 5.5, 4.0 to 5.0, or about 4.5, as measured at 24.3° C.

Generally, an oral rinse composition can include any one more naturally or synthetically derived flavorings, fragrances, sweeteners, etc., in amounts suitable to impart the oral rinse composition with desired organoleptic properties. Preferred flavorings do not adversely affect stability of an oral rinse composition. Flavorings, fragrances, sweeteners, etc., can be added in any useful form, such as a liquid, a powder, etc., and dissolved in solution of an oral rinse composition. Examples of useful flavorings and fragrances include one or more of methyl salicylate, wintergreen, menthol, mint, peppermint, spearmint, fruit flavoring such as berry or citrus, etc. Useful sweeteners include any one or more of sugar alcohols such as erythritol, maltitol, mannitol, sorbitol, xylitol, etc.; sugars such as sucrose, fructose, glucose, etc.; artificial sweeteners such as aspartame, acesulfame potassium, saccharin, sucralose, etc. Sugars such as sucrose, glucose, and fructose are preferably excluded from an oral rinse composition due to the tendency of these compounds to contribute to caries.

The oral rinse composition can include one or more surfactants that act as wetting or solubilizing agents. Examples of useful surfactants include one or more of non-ionic surfactants such as poloxamers, cationic surfactants, anionic surfactants, and amphoteric surfactants. The oral rinse composition can contain essentially no or no amphoteric surfactant. In some embodiments, the oral rinse composition includes poloxamer 407 (for example, sold by BASF as PLURACARE® F 127 Prill NF) as a wetting agent.

The oral rinse composition can have any suitable specific gravity. The specific gravity may for example from 1.0000 to 1.0900, 1.0100 to 1.0800, 1.0200 to 1.0700, 1.0300 to 1.0600, 1.0400 to 1.0500, or 1.041 to 1.053, when measured at 25.0° C.

An oral rinse composition prepared in accordance with the present disclosure generally can provide stability when stored for long periods. An oral rinse composition can provide six months of storage stability when stored in a 7 mL sealed packet constructed of a film (PET/LDPE/Foil/PE/LLDPE), and the sealed packet is placed within a stability chamber at 40° C. and 75% relative humidity for six months. An oral rinse composition can also provide six months of storage stability when stored in the sealed packet and placed within a stability chamber at 25° C. and 60% relative humidity for six months.

An oral rinse composition can also provide six months of storage stability when stored in a 2 oz. cylindrical bottle (HDPE), the bottle is sealed with a foil seal, and the sealed bottle is placed within a stability chamber at 40° C. and 75% relative humidity for six months. The oral rinse composition can also provide six months of storage stability when stored within the sealed bottle and placed within a stability chamber at 25° C. and 60% relative humidity for six months.

Storage stability of the oral rinse composition stored in the sealed packet or the sealed bottle can be demonstrated as any one or more of maintained transparency (colorless or slightly yellow and free of particulates) at six months, stable taste and odor at six months, a total aerobic plate count of less than 100 CFU/g under U.S. Pharmacopeia <61>at six months, a yeast and mold count of less than 10 CFU/g under U.S. Pharmacopeia <61>at six months, and substantial absence of one or more of S. aureus, P. aeruginosa, E. coli, and C. albicans under U.S. Pharmacopeia <62>at six months. The entire contents of U.S. Pharmacopeia <61>and <62>are incorporated herein by reference. Stability may be measured via an evaluation of one or more of the foregoing properties, as determined by user specification. For example, stability can be measured by any one, two, three, four, or five of the following, or all six of the following:

Maintenance of transparency

Stable taste

Stable odor

a total aerobic plate count of less than 100 CFU/g

a yeast and mold count of less than 10 CFU/g

substantial absence of one or more of S. aureus, P. aeruginosa, E. coli, and C. albicans

all evaluated at one, two, three, four, five, six, seven, eight, nine, ten, eleven, or twelve months, or at eighteen months.

Based on difference in weight of an oral rinse composition stored in the sealed packet at time zero and after six months of storage in the stability chamber at 40° C. and 75% relative humidity, the oral rinse composition can demonstrate a moisture increase/loss ranging from −0.12% (moisture increase) to 0.36% (moisture loss). Based on difference in weight of an oral rinse composition stored in the sealed packet at time zero and after six months of storage in the stability chamber at 25° C. and 60% relative humidity, the oral rinse composition can demonstrate a moisture increase/loss ranging from −0.12% (moisture increase) to 0.24% (moisture loss).

Based on difference in weight of an oral rinse composition stored in the sealed bottle at time zero and after six months of storage in the stability chamber at 40° C. and 75% relative humidity, the oral rinse composition can demonstrate a moisture loss ranging from to 0.14 to 0.19%. Based on difference in weight of an oral rinse composition stored in the sealed bottle at time zero and after six months of storage in the stability chamber at 25° C. and 60% relative humidity, the oral rinse composition can demonstrate a moisture loss ranging from 0.07 to 0.11%. The above moisture loss parameters represent other optional measures of stability.

A method of cleansing an oral cavity can generally comprise applying an oral rinse composition to an oral cavity. Application of an oral rinse composition to an oral cavity can advantageously be performed in a single step and provide both cleansing and moisturizing to an oral cavity. The method can comprise administration by a healthcare professional or self-administration. The composition can remain in the oral cavity for any suitable period, such as times ranging from 10 seconds to 1 minute, 15 seconds to 45 seconds, 20 seconds to 40 seconds, 25 seconds to 35 seconds, about 30 seconds, etc.

An oral cleansing kit can include a container and an oral rinse composition provided in a sealed container.

The oral rinse composition can be prepared in any suitable manner such as by combining and mixing components simultaneously or in a designated sequence. One method includes mixing glycerin and a flavoring; adding water and a non-ionic surfactant to the glycerin and the flavoring; mixing the glycerin, flavoring, water, and non-ionic surfactant to form a solution; and dissolving cetylpyridinium chloride in the solution to form an oral rinse composition.

Flavorings may be added to the humectant in a mixing vessel until the flavoring dissolves in the humectant. Generally, any suitable mixing time may be used for dissolving a flavoring in a humectant. Such mixing times can vary depending on conditions such as relative amounts of flavoring and humectant, solubility of flavoring in humectant, etc. but in some cases the mixing time can range from any of 30 seconds to 10 minutes, 1 to 9 minutes, 2 to 8 minutes, 3 to 7 minutes, 4 to 6 minutes, 4.5 to 5.5 minutes, and about 5 minutes. Next, useful amounts of solvent, surfactant, buffering agent/pH adjuster, and sweetener are added. These components can be generally be added in the order listed, any order, simultaneously, etc. These components can then be mixed for any period time such as 10 to 75 minutes, 25 to 65 minutes, 35 to 55 minutes, about 45 minutes, etc. until a uniform solution is produced. An active ingredient is then be added to the solution and further mixed for any suitable time such as 2 to 15 minutes, 4 to 13 minutes, 8 to 11 minutes, about 10 minutes, etc. Additional solvent then can be added and the composition further mixed. Mixing can generally be conducted via devices such as any one or more of a turbine, an impeller, a paddle, injected flow of one or more materials, etc. Utilizing a rotary-type mixing device, mixing speed can range from 10 to 500 revolutions-per-minute (RPM), 100 to 750 RPM, 150 to 350 RPM, 250 to 300 RPM, about 275 RPM, 250 to 650 RPM, 400 to 550 RPM, or any suitable range.

### EXAMPLES

The following examples are nonlimiting.

### Example 1

Oral rinse compositions having the composition shown in Table 1 may be prepared.

TABLE 1

| Ingredient | Function | % w/v | % w/w |
|---|---|---|---|
| Cetylpyridinium chloride, | Active/Anti-gingivitis, | 0.045-0.20 | 0.043-0.19 |
| monohydrate, USP | Anti-plaque | | |
| Purified Water USP | Solvent | 40.00-95.00 | 38.2-90.7 |
| Glycerin USP | Moisturizer-Humectant | 12.00-50.00 | 11.5-47.7 |
| Sodium Citrate, dihydrate USP | Buffering Agent | 0.05-0.30 | 0.05-0.29 |
| Citric acid, anhydrous USP | Buffering Agent/pH | 0.01-0.20 | 0.01-0.19 |
| | adjuster | | |
| Sucralose NF | Sweetening Agent | 0.01-0.20 | 0.01-0.19 |
| Methyl Salicylate NF | Flavoring Agent | 0.005-0.10 | 0.0048-0.095 |
| Poloxamer 407 NF | Wetting and/or solubilizing | 0.006-0.06 | 0.006-0.06 |
| | agent | | |
| Mint Flavoring | Flavoring Agent | 0.005-0.05 | 0.005-0.05 |

### Example 2

Glycerin in the amounts shown in Table 1 added to a mixing vessel. Amounts of methyl salicylate and mint flavoring shown in Table 1 are added to the glycerin and while mixing at about 275 RPM for approximately five minutes, until the methyl salicylate and mint flavoring dissolve in the glycerin. Next, water and the amounts of poloxamer 407, sodium citrate, citric acid, and sucralose shown in Table 1 are added to the vessel in this order. The contents of the vessel are then mixed for approximately 45 minutes at 275 RPM until the mixture is uniform. Cetylpyridinium chloride monohydrate is then added to the vessel and the contents are further mixed at 275 RPM for 10 minutes. Additional water is added to bring the composition to the target amounts (% w/v) shown in Table 1 and the contents of the vessel are further mixed at 275 RPM for 10 minutes.

### Example 3

The composition prepared in accordance with Example 1 is added to a sealed container to form a kit.

Disclosure herein of an ingredient as having one or more functions does not limit that ingredient to having only the disclosed function(s). All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. An oral rinse composition comprising:
a solution including
cetylpyridinium chloride in an amount effective to reduce activity of staphylococcus aureus by at least 99.0% upon contact with the oral rinse composition for 30 seconds in vitro;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

2. The oral rinse composition of claim 1, including cetylpyridinium chloride in an amount effective to reduce activity of porphyromonas gingivalis by at least 99.9998% upon contact with the oral rinse composition for 30 seconds in vitro.

3. The oral rinse composition of claim 1 including 0.095 to 0.105% w/v of cetylpyridinium chloride, based on a total volume of the oral rinse composition.

4. The oral rinse composition of claim 3 including 15.00 to 25.00% w/v of glycerin, based on a total volume of the oral rinse composition.

5. The oral rinse composition of claim 1, the flavoring comprising one or more of mint flavoring and methyl salicylate.

6. The oral rinse composition of claim 1, further comprising one or more of a buffering agent and a pH adjuster.

7. The oral rinse composition of claim 1, further comprising a non-ionic surfactant.

8. The oral rinse composition of claim 1, further comprising poloxamer 407.

9. The oral rinse composition of claim 1 having a pH ranging from 3.5 to 5.5.

10. The oral rinse composition of claim 1, further comprising a sweetener.

11. The oral rinse composition of claim 1 containing essentially no alcohol other than glycerin.

12. The oral rinse composition of claim 1 containing essentially no ethanol.

13. The oral rinse composition of claim 1 containing essentially no hydrogen peroxide.

14. The oral rinse composition of claim 1 containing less than 0.01 w/v of hydrogen peroxide, based on a total volume of the oral rinse composition.

15. An oral cleansing kit comprising:
a container; and
an oral rinse composition in the container, the oral rinse composition comprising solution including
cetylpyridinium chloride in an amount effective to reduce activity of staphylococcus aureus by at least 99.0% upon contact with the oral rinse composition for 30 seconds in vitro;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

16. A method of cleansing an oral cavity, the method comprising:
applying an oral rinse composition to an oral cavity, the oral rinse composition comprising a solution including
cetylpyridinium chloride in an amount effective to reduce activity of staphylococcus aureus by at least 99.0% upon contact with the oral rinse composition for 30 seconds in vitro;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

17. A method of making an oral rinse composition, the method comprising:
mixing glycerin and a flavoring;
adding water and a non-ionic surfactant to the glycerin and the flavoring;
mixing the glycerin, flavoring, water, and non-ionic surfactant to form a solution;
dissolving cetylpyridinium chloride in the solution to form an oral rinse composition;
the oral rinse composition including cetylpyridinium chloride in an amount effective to reduce activity of staphylococcus aureus by at least 99.0% upon contact with the oral rinse composition for 30 seconds in vitro, glycerin in an amount ranging from 15.00 to 45.00% w/v based on a total volume of the oral rinse composition; and water in a minimum amount of 40% w/v based on a total volume of the oral rinse composition; the oral rinse composition comprising essentially no polyhydric alcohol other than glycerin and essentially no amphoteric surfactant.

18. An oral rinse composition comprising:
a solution including
cetylpyridinium chloride in an amount ranging from 0.045 to 0.105% w/v based on a total volume of the oral rinse composition;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

19. The oral rinse composition of claim 18, including 15.00 to 25.00% w/v of glycerin, based on a total volume of the oral rinse composition.

20. The oral rinse composition of claim 18, the flavoring comprising one or more of mint flavoring and methyl salicylate.

21. The oral rinse composition of claim 18, further comprising one or more of a buffering agent and a pH adjuster.

22. The oral rinse composition of claim 18, further comprising a non-ionic surfactant.

23. The oral rinse composition of claim 18, further comprising poloxamer 407.

24. The oral rinse composition of claim 18 having a pH ranging from 3.5 to 5.5.

25. The oral rinse composition of claim 18, further comprising a sweetener.

26. The oral rinse composition of claim 18 containing essentially no alcohol other than glycerin.

27. The oral rinse composition of claim 18 containing essentially no ethanol.

28. The oral rinse composition of claim 18 containing essentially no hydrogen peroxide.

29. The oral rinse composition of claim 18 containing less than 0.01 w/v of hydrogen peroxide, based on a total volume of the oral rinse composition.

30. An oral cleansing kit comprising:
a container; and
an oral rinse composition in the container, the oral rinse composition comprising solution including
cetylpyridinium chloride in an amount ranging from 0.045 to 0.105% w/v based on a total volume of the oral rinse composition;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

31. A method of cleansing an oral cavity, the method comprising:
applying an oral rinse composition to an oral cavity, the oral rinse composition comprising a solution including
cetylpyridinium chloride in an amount ranging from 0.045 to 0.105% w/v based on a total volume of the oral rinse composition;
glycerin in an amount ranging from 15.00 to 45.00% w/v based on a total volume of the oral rinse composition;
flavoring; and
water in a minimum amount of 40% w/v based on a total volume of the oral rinse composition;
the oral rinse composition comprising essentially no polyhydric alcohol other than glycerin and essentially no amphoteric surfactant.

32. A method of making an oral rinse composition, the method comprising:
mixing glycerin and a flavoring;
adding water and a non-ionic surfactant to the glycerin and the flavoring;
mixing the glycerin, flavoring, water, and non-ionic surfactant to form a solution;
dissolving cetylpyridinium chloride in the solution to form an oral rinse composition;
the oral rinse composition including cetylpyridinium chloride in an amount ranging from 0.045 to 0.105% w/v based on a total volume of the oral rinse composition, glycerin in an amount ranging from 15.00 to 45.00% w/v based on a total volume of the oral rinse composition; and water in a minimum amount of 40% w/v based on a total volume of the oral rinse composition; the oral rinse composition comprising essentially no polyhydric alcohol other than glycerin and essentially no amphoteric surfactant.
